(19) United States
(12) Patent Application Publication
(10) Pub. No.: US 2025/0341472 A1
(43) Pub. Date: Nov. 6, 2025

(54) ENHANCED IMAGING & QUANTIFICATION TECHNIQUES FOR LATERAL FLOW ASSAYS

(71) Applicant: HEMEX HEALTH, INC., PORTLAND, OR (US)

(72) Inventors: Peter Galen, Portland, OR (US); James Daren Bledsoe, Portland, OR (US); Alireza Avanaki, Portland, OR (US); Benjamin Holt Bishop, Portland, OR (US); Tyler Witte, Portland, OR (US); Yiyang Fei, Portland, OR (US); John Hinshaw, Portland, OR (US); Matthew Christian Lind, Portland, OR (US)

(21) Appl. No.: 18/860,837

(22) PCT Filed: Apr. 28, 2023
(86) PCT No.: PCT/US23/66405

§ 371 Date: Oct. 28, 2024

Related U.S. Application Data
(60) Provisional application No. 63/363,793, filed on Apr. 28, 2022.

(51) Int. Cl. G01N 21/84; G01N 21/17; G01N 21/77; G01N 33/543

(52) U.S. Cl. CPC G01N 21/8483; G01N 21/17; G01N 21/77; G01N 33/54388; G01N 2021/7759; G01N 2021/7786

(57) ABSTRACT

The disclosed methods and systems use multiple images of a sample test strip used in a lateral flow assay to enhance detection and improve quantification of analyte(s) present in a patient sample. The multiple images are combined into a single image, which is then collapsed into a signal representative of a single 2D image with reduced noise.

### CROSS-REFERENCE TO RELATED APPLICATIONS

This application claims priority and benefit from the U.S. Provisional Patent Application 63/363,793, filed Apr. 28, 2022, and titled, “DISEASE DIAGNOSTIC PLATFORM FOR IMPROVED IMAGING AND QUANTIFICATION,” which is incorporated herein by reference in its entirety for all purposes.

### BACKGROUND

Lateral flow test is a versatile technology widely deployed across medical diagnostics, pharma, environmental testing, animal and plant health, and food testing industries to confirm the presence or absence of target pathogens, analytes, or biomarkers. During a lateral flow test, colored or fluorescent nanoparticle-infused antibodies are mixed with a sample so they can bind to target analytes, if present. The sample is then allowed to flow across a nitrocellulose membrane where it aggregates along a test line or lines if the target biomarker or analyte is present. It also aggregates along a control line that is used to verify the test is working properly and/or to help quantify a test line. An automatic lateral flow test reader quickly and accurately analyzes the color and density of the resulting test and control lines to determine the presence or absence of the target biomarker or analyte and determines the test result to detect a disease or physiological state based on those metrics. The quantity of the target biomarker may also be determined based on the same data.

In some circumstances, detecting the presence of colored or fluorescent biomarkers or analytes at the test and control lines of a lateral flow test can be done visually under natural lighting. Visual reading of lateral flow tests may not be possible for tests requiring fluorescence outside the visible spectrum, which must always be done with a reader. In other circumstances, excitation sources emit light at a wavelength within the visible spectrum but that may be harmful to a human eye, which also requires a reader to prevent injury. Because of these issues, it is often difficult for humans to accurately read fluorescence based lateral flow tests without the aid of a reader, especially when the target biomarker or analyte density is low, when the density must be quantified, or when alternative lighting (such as ultraviolet lighting) is required. The industry would benefit from enhanced imaging and quantification techniques in lateral flow assays.

### BRIEF DESCRIPTION OF THE DRAWINGS

Non-limiting and non-exhaustive embodiments of the invention are described with reference to the following drawings. In the drawings, like reference numerals refer to like parts throughout the various figures, unless otherwise specified, wherein:

FIG. 1 shows a conventional lateral flow assay.

FIG. 2 shows an example process of detecting disease in a lateral flow assay according to the disclosure.

FIGS. 3A-3C show various images of a sample test strip that are analyzed to form signals that have signal characteristics indicative of disease or physiological state.

FIGS. 4A and 4B show examples of background features removal in the image analysis of the sample test strips.

FIG. 5 shows examples of multiple images of a single cartridge with sample tests strips shown in multiple lighting states that include a fluorescent fiducial.

FIG. 6 shows examples of multiple images of a single cartridge with sample test strips shown in multiple lighting states that include timing indicators.

### DETAILED DESCRIPTION

The disclosed lateral flow systems and methods detect and diagnose various disease states including those that use an antibody-antigen binding techniques to detect presence of disease and those detecting an analyte that indicates a disease state. The disclosed lateral flow systems and methods can also detect other physiological states, such as pregnancy and cortisol levels, in a human or animal subject. These novel techniques enhance lateral flow assay imaging and quantification to allow for disease and physiological condition detection and quantification at lower quantities and with higher accuracy. Some of the examples discussed herein are automated lateral flow systems and methods that image a sample test strip to determine whether disease or a physiological condition is present. Automated means systems or methods of detecting disease that automatically analyze an image or images of a sample test strip. The sample test strip can be integrated within a cartridge in some examples. That cartridge could be inserted into a cartridge opening of a reader that positions the cartridge for the image capture to occur before, during, or after the lateral flow assay is run.

These systems and methods capture image(s) of the sample test strip during the run of the lateral flow assay or after the lateral flow assay run is complete for processing. The image(s) are captured by either an automatic event—such as a cartridge with the sample test strip actuating the image capture after it is inserted into a cartridge opening of a reader—or can be captured by a manual trigger—such as a user observing that the sample test strip is ready for image capture. In some cases, the user is unable to observe that the sample is ready for testing, but the automatic event automatically detects that the test is ready for detection and/or quantification by any suitable method. For example, the disclosed systems and methods could capture a “test ready” image that determines that the cartridge is correctly positioned within its reader to then capture the images necessary to process the lateral flow assay test results. After the image capture, the disclosed systems and methods automatically analyze the captured image(s) to determine presence of an analyte or a disease or physiological state based on the analyzed image(s).

In an example, an automatic lateral flow test reader employs image capture, image analysis, and signal analysis techniques in a controlled environment to quickly and accurately detect and quantify presence of marked antibodies in a lateral flow test. This approach to reading a lateral flow test can quickly and accurately determine the test result with improved accuracy and speed compared to the conventional methods. Typically, conventional approaches hold an assay cartridge at a set distance from an optical sensor, such as a camera, and illuminate the cartridge, if needed, for the image capture.

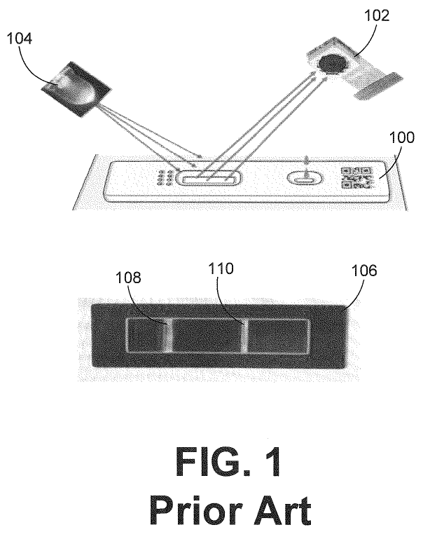

FIG. 1 shows a conventional approach of detecting in a lateral flow assay 100. The lateral flow assay 100 is a sample test strip that is imaged using a camera 102 positioned at an angle with respect to the assay 100. During the image capture, an excitation source emits light, such as UV light, towards the assay 100 to cause excitation of fluorescent conjugated antibodies present in the assay. The assay 100 is imaged 106 to show that portions of the sample with dye conjugated antibodies that are attached to the target analyte at the test line 110 while the remainder of the sample travels to the control line 108 to bind there.

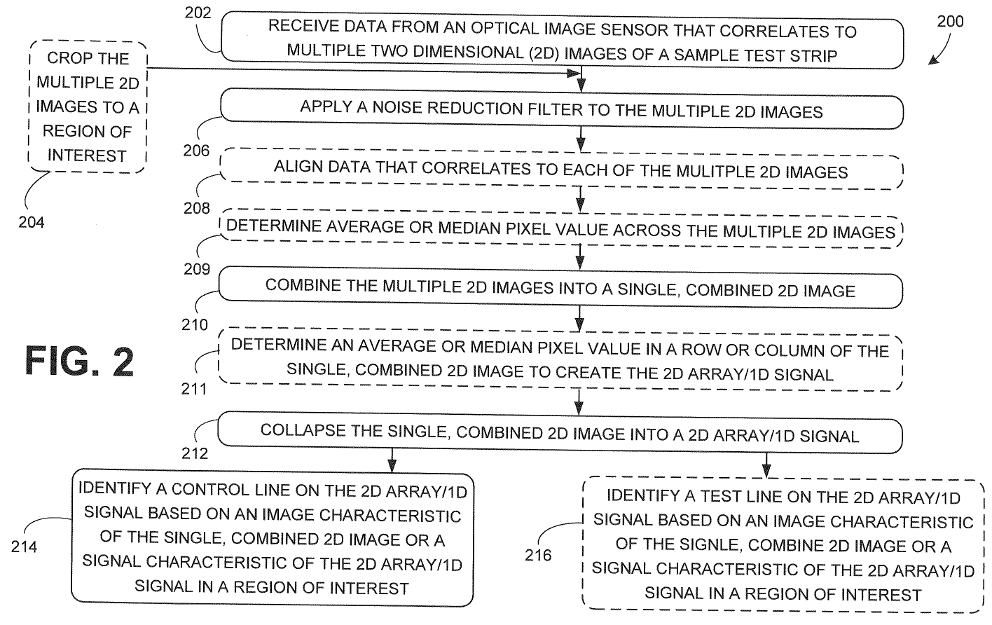

Turning now to FIG. 2, the disclosed systems and methods of detecting disease or a physiological state in a lateral a flow assay 200 improve detection of small volumes of analyte indicating disease or the physiological state present in the sample applied to the test strip and improve the ability to quantify the detected disease or physiological state using image processing techniques. The disclosed systems and methods receive data from an optical image sensor, such as a camera, that correlates to multiple two-dimensional (2D) images of a sample test strip 202. In an example, a 5-megapixel CMOS camera with a Bayer filter is used to image the test strips. The sample test strip has dye-conjugated antibodies, and a patient sample is applied to the sample test strip. The dye conjugated antibodies bind to any analyte present in the patient sample. Some patient samples have analyte—those that are “positive” for the target analyte—while other patient samples do not include the target analyte—those that are “negative” for the target analyte. Depending on the target disease or physiological state, either a positive or a negative result could indicate the target disease or the physiological condition.

In other examples, such as testing for presence of A1C in blood, a test line always appears in which case this physiological condition needs to be analyzed for the quantity of the AIC present in the blood. The target disease can be an infectious disease in some examples or another disease type that is detectable by presence or absence of a target analyte. In other examples, a physiological condition is indicated by the presence or absence or the target analyte. In some cases, the quantification of the target analyte is used to determine if a disease, carrier, or physiological state exists or to determine the severity of disease or physiological condition.

The disclosed methods and systems can capture multiple images at any one or more time(s) during the run of the lateral flow assay or at the completion of the assay run. The image captures can occur on a timed schedule spaced apart by pre-selected time periods, for example. Those pre-selected time periods between image captures can be uniform or can vary if multiple image captures are scheduled. The pre-selected time periods can be timed to occur during or after a time range within which a control line is expected to appear and/or within which a test line is expected to appear on the sample test strip. For example, a control line is expected to appear within 1-2 minutes of commencing the lateral flow assay run while a test line is expected to appear within 3-5 minutes of commencing the lateral flow assay run. Image capture is scheduled at 1, 2, 3, 4, and 5 minutes in this example. If the test line does not yet appear on the captured images at the 5-minute image capture, then additional image captures occur at one or more additional time intervals up to a maximum time period. The maximum time period and the times at which the control line and test line typically appear vary with the target analyte. Another image capture or multiple captures outside of a pre-selected time period or schedule could be actuated by a user or triggered by results indicating further image capture might be helpful in detecting the analyte. In other examples, an image capture may not be necessary if the control line and test line are detected and/or quantified at a previous image capture time.

The disclosed systems and methods capture multiple images of the sample test strip. The multiple images help reduce noise and detect disease with a lower volume of target analyte in the patient sample applied to the test strip. In one example, three or more images of the sample test strip are captured. Other quantities of captured images can be used. The quantity of captured images can vary depending on the target analyte and the time of capture during the assay run. For example, a higher quantity of images could be captured at a region of interest for the test line early in the assay run to filter more noise from the signal being analyzed to detect characteristics of the target analyte than later in the assay run when a higher volume of target analyte (if present) would be likely to be present. Also, a lower quantity of images could be captured after detection of the target analyte at a test line in a region of interest because the target analyte is known to be present. In other examples, the same quantity of images is captured multiple times during the lateral flow assay run to both detect and then quantify the target analyte over time. Alternatively, the same number of images are captured at multiple times during the assay run to either detect or to detect and quantify (one or more times) the target analyte at one or more of the multiple times the images are captured.

In the examples with images captured multiple times during the assay run, multiple images are captured at each time and respectively combined into distinct single 2D images. A first set of images captured at a first time during the assay run are combined into a first signal representative of a first single 2D image. A second set of images captured at a second time during the assay run are combined into a second signal representative of a second single 2D image. A single, combined 2D image is created for each time during the assay run.

After the data correlating to the multiple 2D images is received from the optical image sensor, the images could be cropped to a region of interest 204. The region of interest can be any portion or multiple portions of the image(s). In the disclosed systems and methods, each of the multiple images are cropped to the same region or regions of interest. Alternatively, a portion of the multiple images could be cropped to different regions of interest, which could be helpful to compare results of the between the different image groups to ensure that the fidelity of the chosen region of interest to which all images are cropped.

The region of interest is a portion of the sample test strip in which a lateral flow assay result is expected to appear. In an example, the multiple images can each be cropped to two regions of interest-the first region of interest is the portion of the sample test strip within which a control line is expected to appear, and the second region of interest is the portion of the sample test strip within which a test line is expected to appear. The control line is a line appearing in all lateral flow tests to indicate that the assay run functioned properly. The test line only appears if the target analyte or other compound is present in the sample. That is, for example, the test line indicates a disease state or physiological condition. Oftentimes, the control line and the test line are spaced apart from each other on the sample test strip a designated or pre-defined distance. Multiple test lines may be used to find multiple analytes.

The disclosed systems and methods apply a noise reduction filter to the multiple 2D images 206. For example, the noise reduction filter can be a median or a mean filter or any other digital filtering technique that helps reduce noise in the signal(s) or the image itself and boost the signal of the test and control lines. In some example systems and methods, the data correlating to each of the multiple 2D images is aligned before it is further processed 208. This means that points in the images or on the signals representing the images are aligned with each other in some manner. One example is to align them positionally based on a reference marker on an image, such as a control line or an edge or other marker on the sample test strip. The image or signal alignment can occur before applying the noise reduction filter, in some examples.

Also in some examples, the disclosed systems and methods determine an average or median pixel value across the multiple 2D images 209. The mean or median pixel value for the same pixel location is taken across the multiple 2D images to combine them into a single 2D image 209, in these examples. The multiple 2D images are combined into a single, combined 2D image 210 whether the mean or median pixel value technique is used or another technique.

The mean or median pixel value in each row or column is used to collapse the images because it is the value representing the respective row or column. For example, the values of each pixel in each of the multiple images can be averaged or the median of the pixels can be taken 209 to create the combined, single 2D image 210. In this example, the images are first aligned so that the pixel values of each image match to allow the same pixel in each respective image to be averaged or the median value calculated. Alternative methods to evaluate images or portions of signals representing the images can be used.

The combined, single 2D image can be collapsed into a 2D array or a one-dimensional (1D) signal with varying intensity along the signal line 212 to help reduce temporal noise. The intensity values from multiple images represented by the ID signal lines could be averaged together. To collapse the single, combined 2D image to a 2D array/1D signal, the disclosed systems and methods can determine an average or median pixel value in a row or column of the single, combined 2D image 211. It is this 1D signal from which the control line(s) and test line(s) can be determined by one or more detected signal characteristics. Alternatively, or additionally, the single, combined 2D image 210 can be analyzed to determine a control line or test line by evaluating the intensity or brightness values of the image and comparing them to known data or each other.

Combining multiple 2D images can occur for the multiple images that have certain image characteristic values within a defined difference tolerance, in some examples. The difference tolerance analysis can exclude images with values that exceed a certain threshold, fall outside a tolerated value range, or appear as anomalies with respect to a portion or all of the other images. Some examples of capturing the multiple images have excluded one or more images from the images that are combined into the single 2D image. The exclusion of one or more images can be a result of the image falling outside the difference tolerance or being captured too early, too late, or without proper lighting. One or more images can be excluded for any suitable reason, including to enhance the fidelity of the median or averaged data analyzed in the process of collapsing the median or mean of the pixel values in a row or column of the single, combined 2D image to create the 2D array/1D signal. Such values outside the difference tolerance would skew the median or average values. In other examples, all of the captured images are analyzed to combine into the single 2D image. In this example, each of the capture images is aligned then combined. In some cases, if an image is excluded, an additional image can be collected to replace it.

As explained above, the combined, single 2D image is collapsed along either the x or the y axis to a 2D array or ID signal with intensity values along the signal line. This “collapsing” of the 2D images to a 2D array or 1D signal leaves one positional component (x or y) and the intensity value of the pixel, which helps reduce spatial noise and allows for further signal analysis and quantification.

The disclosed systems and methods also identify a control line on the 2D array/1D signal based on one of the single, combined 2D image characteristics 214 or a signal characteristic of the 1D signal/2D array. In some examples, the image characteristic or signal characteristic is analyzed throughout the image or signal, respectively, or with respect to multiple portions of the single 2D image or 2D array/ID signal. In other examples, the control line is identified by analyzing the image characteristic or signal characteristic in a region of interest of the respective image or signal. As discussed above, the region of choice can be a portion of the image or signal within which the control line is expected to be present.

Some sample test strips have patient samples without the target analyte. In these examples, the absence of the target analyte would produce a result of the control line appearing on the sample test strip without a test line. In other sample test strips, the patient sample includes the target analyte in which case a test line should appear on the sample test strip in addition to and physically separated from the control line. In the examples with a patient sample that includes the target analyte, the disclosed systems and methods also identify a test line on the combined, single 2D image based on an image characteristic 216 that can be the same image or signal characteristic or a different image or signal characteristic than is used to identify the control line. The single 2D image or ID signal used to identify the test line can be the same or a different image as the single 2D image or ID signal used to identify the control line in some examples. If the single 2D image or 1D signal includes both the region of interest for the control line and the region of interest for the test line, then a single 2D image or ID signal can be used to identify both the control line and the test line.

In some examples, the image characteristic upon which the control line and/or the test line are identified is a brightness value of multiple pixels in the combined, single 2D image or 1D signal. To obtain the brightness value, a convolution filter can be applied to the combined, single 2D image or 1D signal in some examples. The brightness value can be a value for multiple pixels in the single 2D image or ID signal within a region of interest. The region of interest in this example could be the region within which the control or test line is expected to be positioned which appears as a contrast to the values of the surrounding background pixels. The brightness values can also be analyzed for each pixel in the single 2D image or each peak in a 1D signal.

The brightness values (or other image or signal characteristic(s) being analyzed to identify the control and/or test lines) can be compared to a known threshold or range of values of control lines and/or test lines that are previously determined to properly indicate the control or test lines, respectively. The brightness value is compared to the known value either from previous values measured in known positive identification of control and/or test lines or from empirical data. The control line and/or test line are identified based on the comparison(s) in this example.

In an example, in order to characterize the behavior of the control line and test line(s) during the assay run, such as the control line's and test line(s)' respective signals' amplitude, width, and band migration speed, the automatic lateral flow test reader uses a camera system to capture one or more images of the lateral flow sample test strip. The sample test strip, as discussed above, can be included in a cartridge that is placed into a positioning tool like a diagnostic assay reader for imaging. The processor either in the tool or reader or remotely analyzes the images to identify and quantify the relative amount of each control line or test line(s). However, if the control line(s) and test line(s) are in different illumination spectrums, then multiple control lines can exist on the same test strip. The process can be broken down into three stages: image capture, image processing and analysis, and signal quantification with result interpretation. The sample tested can be any patient biological sample including blood, tissue, mucus, or the like that can migrate along or be prepared to migrate along the lateral flow assay. The sample tested can also be used in some non-biologic samples and in animal samples.

In the image capture stage, one or more images of the lateral flow test cartridge is taken over time in an enclosed and controlled environment to minimize interference from ambient light sources. Image capture is optimized for such things as white point, exposure, resolution, duration, spectra, and illumination. For example, some colored lateral flow tests lines are most visible under white light while others are best viewed under ultraviolet (UV) lighting or a combination of wavelengths of light. The intensity of the lighting is optimized to maximize visibility of the test line while minimizing background signals. Consideration is required for such things as signal degradation, for example, due to prolonged exposure to UV light. Multiple images of the same cartridge are captured (for example, 3-5 images) and combined using a per-pixel median or mean filter into a single, combined 2D image to reduce image noise and improve the signal-to-noise ratio during the detection and quantification stage. Increasing the number of images typically results in better noise suppression but should be balanced against the increased imaging and processing required for high quantities of images. The image capture system can be calibrated through a feedback loop mechanism where individual parameters in the image capture system are adjusted, then images are captured and analyzed (e.g., using histogram analysis) to determine their impact. Any one or more parameters can be adjusted until an ideal target setting is reached.

In the image processing stage, the captured images are analyzed to identify the region-of-interest (ROI) encompassing the cellulose membrane on the lateral flow cartridge. Depending on the illuminant used for image capture, different color channels may be used for ROI detection. For example, when a conventional CMOS camera with a Bayer filter is used, under UV lighting, the blue channel of the captured images shows the highest contrast between the ROI and background and can therefore be used for ROI detection. Automatic brightness and contrast algorithms may be used to highlight the ROI region. Multiple images may be combined mathematically to reduce noise. One approach to ROI detection is to use image processing techniques, such as thresholding, blurring, gamma-correction, erosion, contouring, and bounding box fitting. Another approach is to use signal processing techniques such as template matching. Results from multiple ROI detection approaches are correlated to accurately identify the ROI and reject errant conditions, such as an incorrectly oriented cartridge or absence of a cartridge. Once the ROI is reliably identified, the ROI region of the image can be further processed to reduce noise and to summarize its contents. Noise reduction can be done by applying a median or mean filter along each column of the image. Since the test and control lines can shift locations within the ROI along a single axis, the distance between the test line and the control line remains the same. The ROI image can be reduced to a single signal row along that axis. The same concept can be applied to other color channels beyond a Bayer filter embodiment described in this example.

In the quantification stage, the signal from the image processing step is analyzed to find peaks within the signal to identify the control and test line positions. Since the distances between the control and test lines are fixed, and the control line is expected to generate a high peak in the signal, it is easy to first identify the control line by finding a prominent peak in the signal within a range of the signal where the control line is expected to exist, then identify the test line(s), if present, at fixed offsets from the control line. The presence of test line signal peaks is directly correlated with the presence or absence of the target analyte indicating a disease state or physiological state. Measuring the test line signal peak amplitude or its integral produces a quantification metric that can be correlated with the density of the target analyte. Sometimes, the relative intensities of the test line(s) and the control lines are important for quantification. By comparing the quantified test line metrics against known thresholds, an interpretive result is determined. Similar quantification metrics generated for the control line can confirm the test is working properly. Noise power and amplitude of the background signal outside the control and test line regions are measured to indicate the quality of the test results so poor tests due to unexpected artifacts can be rejected.

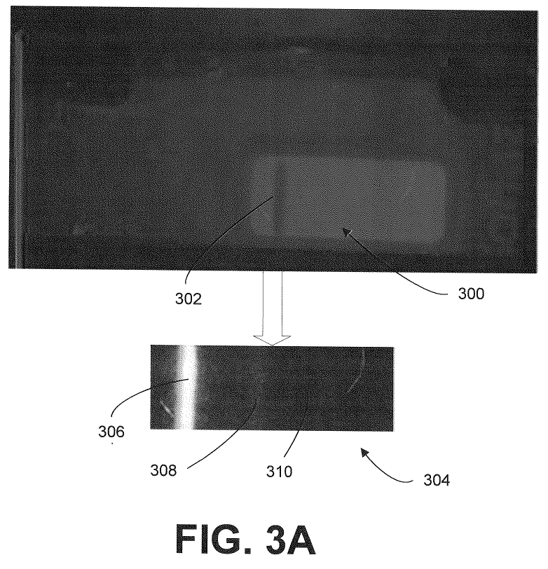

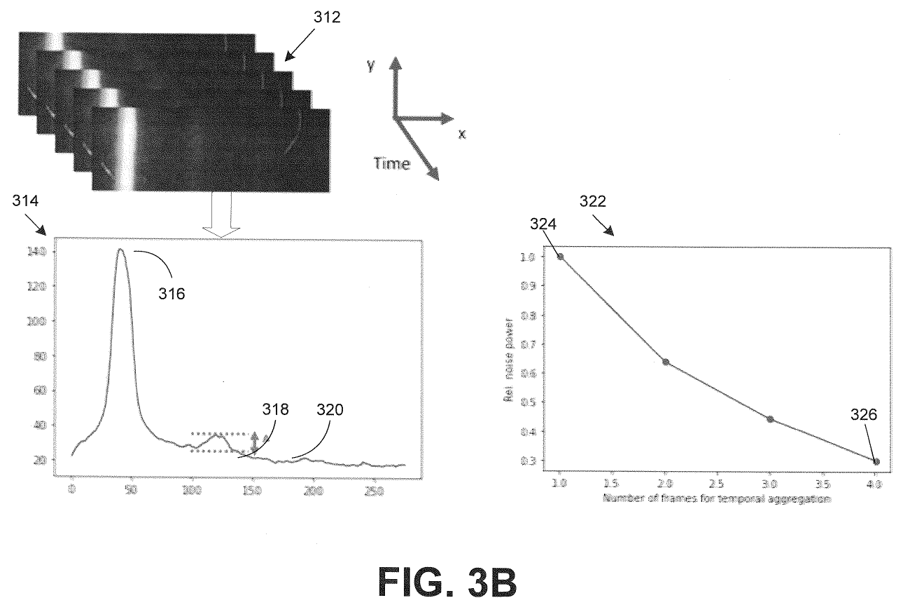

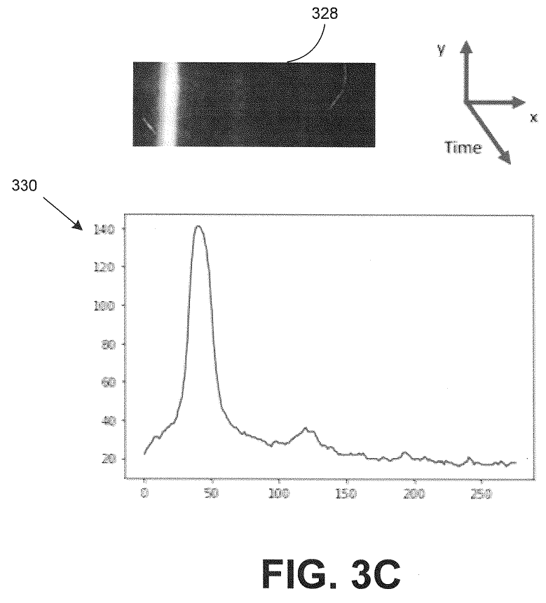

FIGS. 3A-3C shows images of a sample test strip that are analyzed to form signals that have signal characteristics indicative of a disease state. In this example, SARS-COV-2 is the target analyte, but this example can apply to any other infectious or chronic disease, congenital antigen, or physiological state. The sample test strip 300 shown in FIG. 3A is imaged in UV light using an optical image sensor that includes a Bayer filter with RGB channels. The red channel is the channel of interest in this example. The image is cropped to the region of interest 304. In this case, the region of interest (ROI) is a known size. The selected ROI is brighter than the background. The disclosed systems and methods can use template matching to select the ROI. The measured metric is the brightness value difference between the test band 308 and the background 310. The difference in brightness value identifies and quantifies the test line volume of the target analyte, which is SARS-COV-2 in this example. This brightness value metric quantifies the number of viral particles in the SARS-COV-2 sample (or other quantification value for other analytes) based on the intensity of the test line in the captured image(s). Further, this brightness value metric is also used to detect a positive SARS-COV-2 assay run by comparing the detected brightness value to a threshold above the brightness value metrics known for negative samples.

Referring now to FIG. 3B, multiple images 312 are cropped and processed. The multiple images 312 are captured over a period of time, which creates temporal redundancy. The cropped multiple images 312 have a width greater than one pixel, which also creates spatial redundancy. The brightness values are aggregated over the cropped region of interest over time to create the representative signal 314 of the aggregated multiple images of brightness intensity values on the y-axis over the spatial positions on the test strip on the x-axis. A control line peak 316 represents the brightness values of the control line while the test line peak 318 represents the brightness values of the test line.

In this example, five images 312 are aggregated together into a combined 2D image, then “collapsed” to a ID signal with intensity values along the signal line or a 2D array to reduce the noise of any individual captured image. The spatial aggregation occurs by determining the median brightness intensity values of the control line and the test line over the cartridge width while the temporal aggregation occurs by averaging the brightness intensity values of the control line, background, and the test line over time. This reduces the noise power, which smooths the signal to allow for the detection of a weaker test line signal 318 that would not be detectable in a signal with higher noise power. The graph showing the relative noise power (y-axis) to the number of frames for temporal aggregation (x-axis) 322 indicates a ˜70% reduction in noise power from a single image value of 1.0 to the value of 0.3 using 4.0 images.

The noise power present in a single image 328 could drown a weak test line signal 330, such as the example shown in FIG. 3C. The signal 328 representative of only a single 2D image shows a high quantity of irregular or “noisy” signal variations than the multiple image signal 314 shown in FIG. 3B. Noisier signals can drown the detectability of the test line peak 318 altogether.

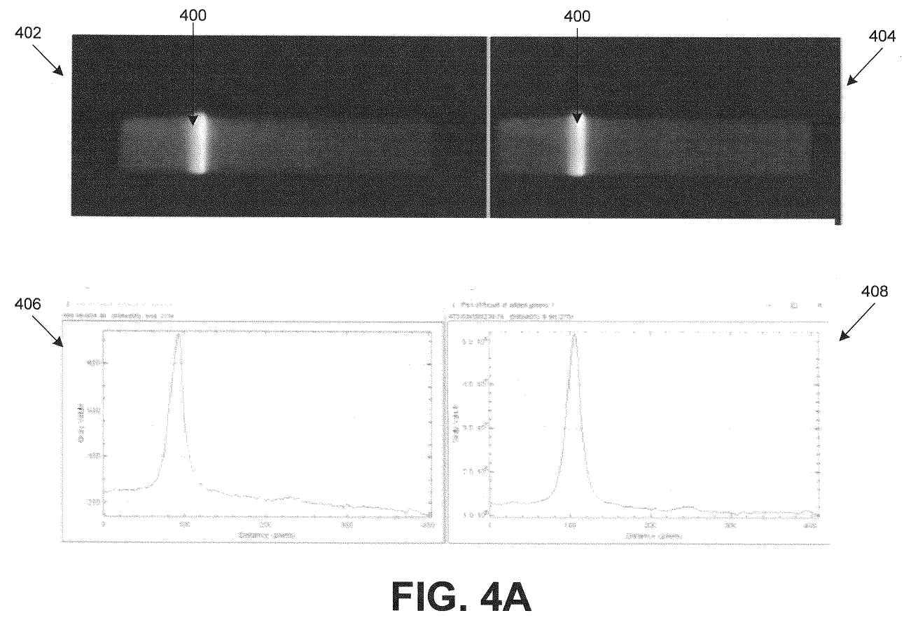

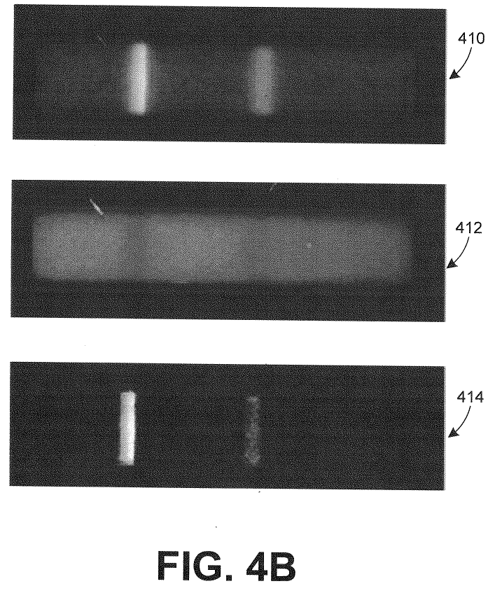

FIGS. 4A and 4B show examples of background features removed in the image analysis of the sample test strips. Some energy sources outside the primary measuring source cause background features in the captured images. For example, primary measurements are taken in the red-light spectrum in the image while background features can be detected in the green light spectrum in the image. For example, background features detected in other energy spectra indicate presence of system or component variations as described above. By detecting these in other spectra, the background features are removed from the primary measurements. These background features can be subtracted or otherwise mathematically combined with the primary spectrum in order to remove false or confounding features and reveal the true signal. Background features may also be detected in energy spectra from other sources, such as acoustic energy.

FIGS. 4A and 4B show examples of removing background features and increasing the detectable signal using secondary and tertiary energy spectra. In FIG. 4A, the images 402, 404 show a control line 400, but the test lines are invisible (either as being a low volume of target analyte or simply not present in the sample) without reducing the background noise and analyzing the signal characteristics of their respective signals 406, 408. The image 402 on the left represents the primary spectrum (red channel data in this example), which has its multiple images averaged for their respective brightness intensity values. The image 404 on the right represents the image on the left after both the primary and secondary (green channel data in this example) information are incorporated together. The primary signal is more clearly distinguished from background features in the right graph.

Alternatively, background features can be removed by increasing signal amplitude or otherwise enhancing the signal to differentiate between the signal and the background. Photobleaching is another method of background reduction. For example, the substrate on which the test and control lines are detected may fluoresce to some degree at the wavelengths of interest. Fluorescent molecules lose their ability to fluoresce with increased exposure to the fluorescent excitation wavelength. The rate at which these molecules lose their ability to fluoresce can depend on the fluorescent molecule. For example, the background may lose its fluorescent capabilities at a faster rate than the test lines(s) and control line(s). Therefore, to reduce the background and increase the signal to background ratio the substrate can be intentionally exposed to the excitation source before or during image capture to increase the limit of detection and accuracy of the fluorescent test.

Further, in a lateral flow test, the excitation source may be undetectable by the camera or filtered out either digitally or physically such that the excitation signal is not detectable by the optical sensor. In these cases, the position of the excitation source may then be placed such that the excitation and response signal are aligned to maximize the response signal. For example, in a fluorescent test, the excitation source may be placed directly behind the test strip to maximize the fluorescent response of the test and control line(s). In the case of a visual test, backlighting and/or front lighting the test strip may also be advantageous. For example, the excitation source may be a green LED that passes through a nitrous cellulose strip, but the test and control line(s) may be bound with a molecule that is known to absorb green light. Backlighting becomes particularly advantageous if the test and control line(s) have a higher rate of reflection than transmission in the range of the signal. Front lighting becomes particularly advantageous if the test and control line(s) have a lower rate of reflection than transmission in the range of the signal. In addition to the reflection and transmission properties of the test and control line(s), the reflection and transmission properties of the fluid within which the sample is mixed and the substrate on which the sample mixture is deposited can lead to backlighting or front lighting providing the best signal to noise ratio, depending on the circumstances.

One example is to incorporate information from energy sources outside of the primary measuring source, which responds to the target analyte. For example, if the primary measurements take place in the red-light spectrum, the target analyte absorbs in the green spectrum. The target analyte response can be positive, for example, causing energy emission, or negative, for example, causing energy absorption. The two or more signals (primary and secondary, etc.) can be combined mathematically or logically to increase target signal amplitude above background levels (signal to noise). Background features may also be detected in energy spectra from other sources, such as acoustic energy or heat energy. For example, FIG. 4B shows an image measured in the red-light spectrum 410, an image measured in the green light spectrum 412, and an image with the red minus the green light spectrum 414. The image with the red minus green light spectrum 414 illustrates a greater intensity difference between the control and test lines and the background.

At lower signal levels (lower levels of analyte), background features (noise) begin to mask or otherwise confuse the signal. For example, background features can be caused by mechanical imperfections, illumination setup, material variations in substrate, and other component or system variations, which produce detectable features in the sensing apparatus. These component or system variations may arise due to limitations in the manufacturing process, inherent limitations in the materials, sample preparation differences from test to test, common or acute wear and tear on the sensing apparatus, environmental effects, interference and specificity issues with the conjugated antibody, or other changes which produce detectable features. Increasing illumination intensity increases both signal and background features. In general, the amplitudes of signal and background increase at differing rates from one another. Because signal and background amplitudes have different responses to illumination, there can be an optimal illumination brightness, which maximizes the difference between the signal and the background. For example, the brightness intensity can be swept from low to high or the signal intensity and background can be monitored. The illumination intensity that produces the greatest difference between signal and background (signal to noise ratio (SNR)) is chosen. The intensity on the target sample can also be increased, for example, by adjusting focus of the excitation energy source, directing the energy source toward the target, or increasing the power to excitation energy source.

Another embodiment changes the illumination wavelength to either increase or decrease both the signal and/or the background features. In general, the amplitudes of the signal and background change at differing rates from one another compared to the illumination wavelength. Because signal and background amplitudes have different responses to wavelength, there can be an optimal illumination wavelength that maximizes the difference between the signal and the background. The illumination wavelengths can be swept while monitoring the signal intensity and background. The illumination wavelength can be chosen that produces the greatest difference between the signal and the background (SNR). For example, multiple illumination sources (such as multiple LEDs, lamps, etc.), broad band spectrum illumination, or multiple filter sets can be used. Further, if the illumination source is used for fluorescing Europium, for example, or a similar material that has a long persistence for fluorescence, then a series of short light flashes reduce background light while maintaining the fluorescent signal.

In some embodiments, identifying the region of interest can be difficult because the system cannot be completely visualized using only fluorescence, which could result in a shifted region of interest, the wrong size region of interest, or not detecting a region at all. Detecting the region of interest is important to ensure that results are only provided if the sample test strip is imaged properly. In some examples, correct positioning of the cartridge within the reader is important to ensure that the sample test strip in the cartridge is in a proper position to be illuminated, imaged, and viewed correctly. Finding this region of interest exactly is important, particularly for sample test strips that can test for multiple analytes.

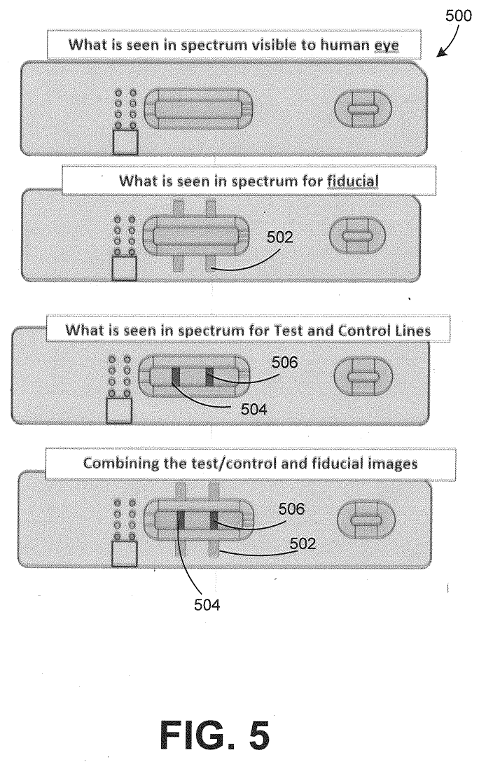

Identifying specific locations for a region of interest is difficult in an imaging system. An example imaging system can include cameras, photodiode arrays, film, and photographic paper. FIG. 5 shows an example cartridge 500 with a fluorescent fiducial 502 in fixed locations relative to points of interest. In this example, the points of interest are the control line 504 and the test line 506. The fluorescent fiducial 502 can respond to a single wavelength or could respond differently at different wavelengths of illumination. When illuminated, the fluorescent fiducial 502 identifies specific locations with high accuracy. The presence, shape, and pattern of these fiducials can be used to identify authentic cartridges. The region of interest can be specified using fiducials to identify key features as well. One example of the use of fluorescent fiducials is to indicate where the algorithm should look for test and control lines. Rather than identifying a rectangle, fitting a template to it, and then looking for the control line 504 and the test line 506, the disclosed methods and systems shown in FIG. 5 have indicators that show exact locations (or regions of interest) for the test line 506 and the control line 504. The images are cropped to include both of those portions of the image and the disclosed systems and methods evaluate the image near the areas indicated by the fluorescent fiducials 502. Other or additional indicators of a region of interest of expected regions of interest can also be used in alternative examples.

The sample test strip can also be authenticated by analyzing the combined 2D image or one or more of the individual images that are combined to create the single 2D image. In the image, an authentication characteristic is detected on the sample test strip to authenticate it. If the authentication characteristic is not present, then the disclosed systems and methods can reject further processing of the sample test strip. If the sample test strip is integrated within a cartridge, the disclosed systems and methods could also eject the cartridge from the reader into which it was inserted for processing. Example authentication characteristics can include visual indicators—such as a unique identifier, image, or color—or mechanical indicators—such as notches or keys molded into a cartridge.

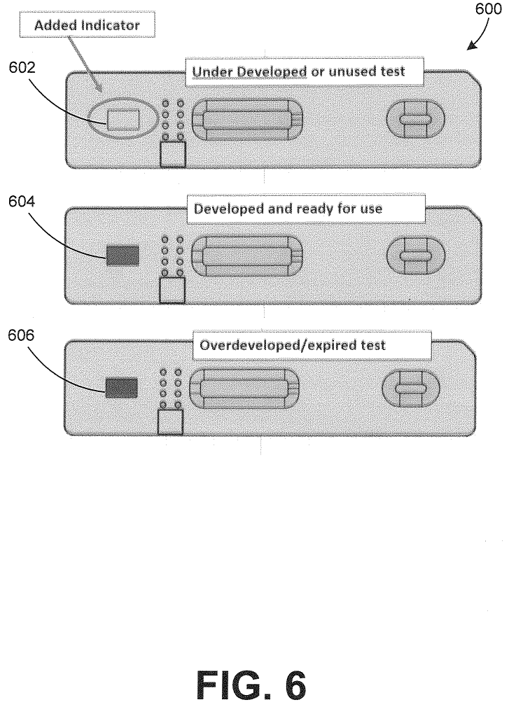

Certain processes require timing or actions to be performed in a certain time range. Timing is important to ensure consistent and accurate results in these tests. For example, a process can be performed too early or too late, which can lead to poor results. If the test is imaged too soon, then the sample may not have flowed long enough, which causes a false negative. If the sample flows too long, back flow can damage or distort the image, which can cause a false positive. FIG. 6 shows an example of a visual indicator that is added to a sample test strip integrated within a cartridge to indicate when it is an acceptable time to perform a process via a light emission change. An underdeveloped or unused test cartridge 600 is shown with a visual indicator 602. The visual indicator is a window in this example that can change from one state (color, pattern, etc.) to another state when the test is ready to be used 604, expired or overdeveloped 606, or the like.

In an example, the visual indicator on the cartridge starts out blue and, when a process such as sample application, is performed correctly, an indicator changes to another color to let the device and/or user know that it is time to run the test. Other examples of processes involving timing are when a cartridge is opened it can only sit for a certain time before being used or can only be read a certain time after the assay run is complete. The visual indicators can be in the fluorescent spectrum far away from target signals to reduce unwanted background or can be in the visible spectrum so users can correctly identify process timing. For example, a visual indicator can be a chemical reaction, a reaction to the patient sample, or a reaction to the extended presence of oxygen. Mechanical interaction, for example, to the presence of increased saturation level from patient sample can also be used.

The subject matter of embodiments disclosed herein is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

What is claimed is:

1. A method of detecting disease or a physiological state in a lateral flow assay, comprising:
receiving data from an optical image sensor that correlates to multiple two-dimensional (2D) images of a sample test strip;
applying a noise reduction filter to each of the respective multiple 2D images to create a single, combined 2D image;
collapsing the single, combined 2D image into a one-dimensional (1D) signal representative of the single, combined 2D image; and
identifying a control line on the 1D signal based on a signal characteristic of the 1D signal.

2. The method of claim 1, further comprising receiving data from the optical image sensor that correlates to three or more 2D images of the sample test strip.

3. The method of claim 1, further comprising aligning data that correlates to each of the multiple 2D images before applying the noise reduction filter to the 2D multiple images.

4. The method of claim 1, wherein applying the noise reduction filter includes applying a median filter.

5. The method 1, further comprising determining an average or a median pixel value across the multiple 2D images to create the single, combined 2D image.

6. The method of claim 1, wherein identifying the control line on the 1D signal includes applying a convolution filter to the 1D signal.

7. The method of claim 1, wherein the signal characteristic of the 1D signal correlates to a brightness value of multiple pixels in the single, combined 2D image.

8. The method of claim 1, wherein the signal characteristic of the 1D signal correlates to a brightness value of each pixel in the single, combined 2D image.

9. The method of claim 1, further comprising identifying a test line on the 1D signal based on the signal characteristic of the 1D signal.

10. The method of claim 9, further comprising:
comparing the signal characteristic used to identify the test line with a known test signal characteristic value indicative of the test line; and
identifying the test line on the single 2D image based on the compared signal characteristic to the known test signal characteristic value.

11. The method of claim 1, further comprising:
comparing the signal characteristic used to identify the control line with a known control signal characteristic value indicative of the control line; and
identifying the control line based on the 1D signal based on the compared signal characteristic to the known control signal characteristic value.

12. The method of claim 1, further comprising photobleaching the sample test strip before the multiple 2D images of the sample test strip are captured.

13. The method of claim 1, further comprising capturing the multiple 2D images during or after a run of the sample assay.

14. The method of claim 1, further comprising capturing the multiple 2D images multiple times at different times during or after a run of the sample assay.

15. The method of claim 14, wherein applying the noise reduction filter to the multiple 2D images captured at the multiple, different times during or after the run includes all of the multiple images captured at the multiple times throughout the run.

16. The method of claim 15, wherein the multiple images captured at a first of the multiple, different times are collapsed into a first 1D signal, and the multiple images captured at a second of the multiple, different times are collapsed into a second 1D signal.

17. The method of claim 1, further comprising backlighting the sample test strip before capturing the multiple 2D images of the sample test strip.

18. The method of claim 1, further comprising front lighting the sample test strip before capturing the multiple 2D images of the sample test strip.

19. The method of claim 1, further comprising:
identifying an authentication characteristic of the sample test strip on the single, combined 2D image or one of the multiple 2D images; and
authenticating the sample test strip based on the identified authentication characteristic.

20. The method of claim 1, further comprising:
capturing an authentication image of the sample test strip;
identifying an authentication characteristic in the authentication image; and
authenticating the sample test strip based on the identified authentication characteristic.

21. The method of claim 1, further comprising:
capturing the multiple 2D images of the sample test strip; and
cropping the multiple 2D images to a region of interest before applying the noise reduction filter to the multiple 2D images.

22. The method of claim 21, wherein cropping the multiple 2D images to the region of interest includes:
identifying a fluorescent fiducial of the control line; and
targeting the cropping of the multiple 2D images to the fluorescent fiducial of the control line.

23. The method of claim 1, further comprising capturing the multiple 2D images using multiple channels of the optical image sensor.

24. The method of claim 23, wherein identifying the control line on the 1D signal includes evaluating data from at least two of the multiple channels of the optical image sensor.

25. The method of claim 24, wherein identifying the control line on the 1D signal includes evaluating control line data from a first of the multiple channels and evaluating background data from a second of the multiple channels.

26. The method of claim 25, further comprising removing the background data from the control line data to create enhanced control line data.

27. The method of claim 25, further comprising combining the background data and the control line data to create enhanced control line data.

28. The method of claim 1, further comprising:
identifying an enhancement characteristic in the 1D signal; and
enhancing the 1D signal by adjusting an aspect of capturing the multiple 2D images based on the enhancement characteristic.

29. The method of claim 28, wherein the enhancement characteristic is an illumination brightness or an illumination wavelength of light used during capture of the multiple 2D images.

30. A system of detecting disease or a physiological state in a lateral flow assay, comprising:
a processor configured to:
receive data from an optical image sensor that correlates to multiple two-dimensional (2D) images of a sample test strip;
apply a noise reduction filter to each of the respective multiple 2D images to create a single, combined 2D image;
collapse the single, combined 2D image into a one-dimensional (1D) signal representative of the single, combined 2D image; and
identify a control line on the 1D signal based on a signal characteristic of the 1D signal.

31. The system of claim 30, wherein the processor is further configured to receive data from the optical image sensor that correlates to three or more 2D images of the sample test strip.

32. The system of claim 30, wherein the processor is further configured to align data that correlates to each of the multiple 2D images before applying the noise reduction filter to the 2D multiple images.

33. The system of claim 30, wherein the processor is further configured to apply the noise reduction filter by applying a median filter.

34. The system of claim 30, wherein the processor is further configured to determine an average or a median pixel value across the multiple 2D images to create the single, combined 2D image before collapsing the 1D signal based on the signal characteristic.

35. The system of claim 30, wherein the processor is further configured to apply a convolution filter to the 1D signal to identify the control line.

36. The system of claim 30, wherein the signal characteristic of the 1D signal includes a brightness or intensity value of multiple pixels in the single, combined 2D image.

37. The system of claim 30, wherein the signal characteristic of the 1D signal includes a brightness or intensity value of each pixel in the single, combined 2D image.

38. The system of claim 30, wherein the processor is further configured to identify a test line on the 1D signal based on the signal characteristic.

39. The system of claim 38, wherein the processor is further configured to:
compare the signal characteristic used to identify the test line with a known test signal characteristic value indicative of the test line; and
identify the test line on the 1D signal based on the compared signal characteristic to the known test signal characteristic value.

40. The system of claim 30, wherein the processor is further configured to:
compare the signal characteristic used to identify the control line with a known control signal characteristic value indicative of the control line; and
identify the control line on the 1D signal based on the compared signal characteristic to the known control signal characteristic value.

41. The system of claim 30, wherein the processor is further configured to capture the multiple 2D images during or after a run of the sample assay.

42. The system of claim 30, wherein the processor is further configured to capture the multiple 2D images multiple times at different times during or after a run of the sample assay.

43. The system of claim 42, wherein the processor is further configured to apply the noise reduction filter to the multiple 2D images captured at the multiple, different times during or after the run includes all of the multiple images captured at the multiple times throughout the run.

44. The system of claim 43, wherein the processor is further configured to collapse the single, combined image representative of a first of the multiple, different times into a first 1D signal, and to collapse the single, combined image representative of a second of the multiple, different times into a second 1D signal.

45. The system of claim 30, wherein the processor is further configured to:
identify an authentication characteristic of the sample test strip on the single, combined 2D image or one of the multiple 2D images; and
authenticate the sample test strip based on the identified authentication characteristic.

46. The system of claim 30, wherein the processor is further configured to:
capture an authentication image of the sample test strip;
identify an authentication characteristic in the authentication image; and
authenticate the sample test strip based on the identified authentication characteristic.

47. The system of claim 30, wherein the processor is further configured to:
capture the multiple 2D images of the sample test strip; and
crop the multiple 2D images to the region of interest before applying the noise reduction filter to the multiple 2D images.

48. The system of claim 47, wherein the processor is further configured to:
crop the multiple 2D images to the region of interest by identifying a fluorescent fiducial of the control line; and
target the cropping of the multiple 2D images to the fluorescent fiducial of the control line.

49. The system of claim 30, wherein the processor is further configured to capture the multiple 2D images using multiple channels of the optical image sensor.

50. The system of claim 49, wherein the processor is further configured to identify the control line on the 1D signal by evaluating data from at least two of the multiple channels of the optical image sensor.

51. The system of claim 50, wherein the processor is further configured to identify the control line on the 1D signal by evaluating control line data from a first of the multiple channels and evaluating background data from a second of the multiple channels.

52. The system of claim 51, wherein the processor is further configured to remove the background data from the control line data to create enhanced control line data.

53. The system of claim 52, wherein the processor is further configured to combine the background data and the control line data to create enhanced control line data.

54. The system of claim 30, wherein the processor is further configured to:
identify an enhancement characteristic in the 1D signal; and
enhance the 1D signal by adjusting an aspect of capturing the multiple 2D images based on the enhancement characteristic.

55. The system of claim 54, wherein the enhancement characteristic is an illumination brightness or an illumination wavelength of light used during capture of the multiple 2D images.
